SEQUENCE LISTING (24 sequences)

SEQ ID NO: 1 - protein, 10 aa, Mus musculus
GYTFTSYWMH

SEQ ID NO: 2 - protein, 17 aa, Mus musculus
YINPTTGYTDYNQKFKD

SEQ ID NO: 3 - protein, 13 aa, Mus musculus
EIYFGDVKTWFAY

SEQ ID NO: 4 - protein, 11 aa, Mus musculus
STSQDISNYLN

SEQ ID NO: 5 - protein, 7 aa, Mus musculus
TSNLQSG

SEQ ID NO: 6 - protein, 9 aa, Mus musculus
QQYSKFPYT

SEQ ID NO: 7 - protein, 10 aa, Mus musculus
GYTFSNYWIE

SEQ ID NO: 8 - protein, 17 aa, Mus musculus
EILPGSDITNYNEKFKD

SEQ ID NO: 9 - protein, 13 aa, Mus musculus
VYYYPGSLAWFAY

SEQ ID NO: 10 - protein, 11 aa, Mus musculus
SASQDISNYLN

SEQ ID NO: 11 - protein, 7 aa, Mus musculus
YTSSLHS

SEQ ID NO: 12 - protein, 9 aa, Mus musculus
QQYSKLPYT

SEQ ID NO: 13 - protein, 10 aa, Mus musculus
GYTFSSYWIE

SEQ ID NO: 14 - protein, 17 aa, Mus musculus
EILPGSGNTYYNEKFKG

SEQ ID NO: 15 - protein, 12 aa, Mus musculus
WEWLLRLYGLDY

SEQ ID NO: 16 - protein, 15 aa, Mus musculus
KASQSVDYDGESYMN

SEQ ID NO: 17 - protein, 7 aa, Mus musculus
AASNLES

SEQ ID NO: 18 - protein, 9 aa, Mus musculus
QQSNEDPYT

SEQ ID NO: 19 - protein, 10 aa, Mus musculus
GFSFSDYFMY

SEQ ID NO: 20 - protein, 17 aa, Mus musculus
TISDGGIYTYYPDSVKG

SEQ ID NO: 21 - protein, 11 aa, Mus musculus
DLDYFGSTIAY

SEQ ID NO: 22 - protein, 14 aa, Mus musculus
RSSAGALTTRNYAN

SEQ ID NO: 23 - protein, 7 aa, Mus musculus
GTSNRAP

SEQ ID NO: 24 - protein, 9 aa, Mus musculus
ALWYSNHWV

(19) United States
(12) Patent Application Publication
(10) Pub. No.: US 2026/0226140 A1
(43) Pub. Date: Aug. 6, 2026

(54) ANTI-SARS-COV-2 MONOCLONAL ANTIBODY AND IMMUNOASSAY METHOD AND IMMUNOASSAY INSTRUMENT FOR SARS-COV-2 USING SAME

(71) Applicant: Denka Company Limited, Tokyo (JP)

(72) Inventors: Toru YAMAGUCHI, Gosen-shi (JP); Daisuke KATO, Gosen-shi (JP); Shino MURAMATSU, Gosen-shi (JP)

(73) Assignee: Denka Company Limited, Tokyo (JP)

(21) Appl. No.: 19/151,947

(22) PCT Filed: Feb. 7, 2024
(86) PCT No.: PCT/JP2024/003985

§ 371 Date: Jul. 29, 2025

(30) Foreign Application Priority Data
Feb. 8, 2023 (JP) 2023-017393

(51) Int. Cl. C07K 16/104; G01N 33/569

(52) U.S. Cl. CPC C07K 16/104; G01N 33/56983; C07K 2317/565; G01N 2333/165; G01N 2469/10; G01N 2470/04

(57) ABSTRACT

Disclosed are a monoclonal antibody capable of rapidly, simply, and highly sensitively detecting and measuring SARS-CoV-2 contained in a test sample, and an immunoassay method and immunoassay device for SARS-CoV-2 using the same. The monoclonal antibody or its antigen-binding fragment includes heavy chain CDR1-CDR3 having specific amino acid sequences and light chain CDR1-CDR3 having specific amino acid sequences. The immunoassay method for SARS-CoV-2 includes performing an immunoassay of SARS-CoV-2 utilizing an antigen-antibody reaction between this monoclonal antibody or its antigen-binding fragment and SARS-CoV-2 in a sample.

### TECHNICAL FIELD

The present invention relates to an anti-SARS-CoV-2 monoclonal antibody, and an immunoassay method and immunoassay device for SARS-CoV-2 using the same.

### BACKGROUND ART

SARS-CoV-2 is a virus belonging to the Coronaviridae family, having a single-stranded positive-sense RNA viral genome. In March 2020, the infectious disease (COVID-19) caused by SARS-CoV-2 became a pandemic, resulting in many infections worldwide. To date in 2022, vaccines have been developed in various countries, and vaccination is progressing globally, but due to the emergence of mutant strains, there is no clear end in sight for the pandemic.

COVID-19 infection is diagnosed using PCR methods and antigen test methods for SARS-CoV-2. As a method for rapidly and easily detecting SARS-CoV-2, immunochromatography methods using anti-SARS-CoV-2 antibodies have been developed, but with no end in sight for the pandemic, more accurate tests are required.

### PRIOR ART DOCUMENTS

### Patent Documents

PATENT DOCUMENT 1 WO 2021/181994

### SUMMARY OF INVENTION

### Problem to be Solved by the Invention

Currently produced monoclonal antibodies against SARS-CoV-2 do not have sufficient sensitivity for detecting SARS-CoV-2, and there is a demand for monoclonal antibodies that react with higher sensitivity.

An object of the present invention is to provide a monoclonal antibody capable of rapidly, simply, and highly sensitively detecting and measuring SARS-CoV-2 contained in a test sample, and an immunoassay method and immunoassay device for SARS-CoV-2 using the same. [Means for Solving the Problems]

As a result of intensive studies on the above problems, the present inventors found specific monoclonal antibodies that react with high sensitivity to SARS-CoV-2, thereby completing the present invention.

That is, the present invention provides the following.
(1) A monoclonal antibody or an antigen-binding fragment thereof, comprising heavy chain CDR1-CDR3 of the following (a-1) to (c-1) and light chain CDR1-CDR3 of the following (d-1) to (f-1). (a-1) heavy chain CDR1 consisting of the amino acid sequence of GYTFTSYWMH (SEQ ID NO: 1),
(b-1) heavy chain CDR2 consisting of the amino acid sequence of YINPTTGYTDYNQKFKD (SEQ ID NO: 2),
(c-1) heavy chain CDR3 consisting of the amino acid sequence of EIYFGDVKTWFAY (SEQ ID NO: 3),
(d-1) light chain CDR1 consisting of the amino acid sequence of STSQDISNYLN (SEQ ID NO: 4),
(e-1) light chain CDR2 consisting of the amino acid sequence of TSN LQSG (SEQ ID NO: 5),
(f-1) light chain CDR3 consisting of the amino acid sequence of QQYSKFPYT (SEQ ID NO: 6)
(2) A monoclonal antibody or an antigen-binding fragment thereof, comprising heavy chain CDR1-CDR3 of the following (a-2) to (c-2) and light chain CDR1-CDR3 of the following (d-2) to (f-2). (a-2) heavy chain CDR1 consisting of the amino acid sequence of GYTFSNYWIE (SEQ ID NO: 7),
(b-2) heavy chain CDR2 consisting of the amino acid sequence of EILPGSDITNYNEKFKD (SEQ ID NO: 8),
(c-2) heavy chain CDR3 consisting of the amino acid sequence of VYYYPGSLAWFAY (SEQ ID NO: 9),
(d-2) light chain CDR1 consisting of the amino acid sequence of SASQDISNYLN (SEQ ID NO: 10),
(e-2) light chain CDR2 consisting of the amino acid sequence of YTSSLHS (SEQ ID NO: 11),
(f-2) light chain CDR3 consisting of the amino acid sequence of QQYSKLPYT (SEQ ID NO: 12)
(3) A monoclonal antibody or an antigen-binding fragment thereof, comprising heavy chain CDR1-CDR3 of the following (a-3) to (c-3) and light chain CDR1-CDR3 of the following (d-3) to (f-3). (a-3) heavy chain CDR1 consisting of the amino acid sequence of GYTFSSYWIE (SEQ ID NO: 13),
(b-3) heavy chain CDR2 consisting of the amino acid sequence of EILPGSGNTYYNEKFKG (SEQ ID NO: 14),
(c-3) heavy chain CDR3 consisting of the amino acid sequence of WEWLLRLYGLDY (SEQ ID NO: 15),
(d-3) light chain CDR1 consisting of the amino acid sequence of KASQSVDYDGESYMN (SEQ ID NO: 16),
(e-3) light chain CDR2 consisting of the amino acid sequence of AASNLES (SEQ ID NO: 17),
(f-3) light chain CDR3 consisting of the amino acid sequence of QQSNEDPYT (SEQ ID NO: 18)
(4) A monoclonal antibody or an antigen-binding fragment thereof, comprising heavy chain CDR1-CDR3 of the following (a-4) to (c-4) and light chain CDR1-CDR3 of the following (d-4) to (f-4). (a-4) heavy chain CDR1 consisting of the amino acid sequence of GFSFSDYFMY (SEQ ID NO: 19),
(b-4) heavy chain CDR2 consisting of the amino acid sequence of TISDGGIYTYYPDSVKG (SEQ ID NO: 20),
(c-4) heavy chain CDR3 consisting of the amino acid sequence of DLDYFGSTIAY (SEQ ID NO: 21),
(d-4) light chain CDR1 consisting of the amino acid sequence of RSSAGALTTRNYAN (SEQ ID NO: 22),
(e-4) light chain CDR2 consisting of the amino acid sequence of GTSNRAP (SEQ ID NO: 23),
(f-4) light chain CDR3 consisting of the amino acid sequence of ALWYSNHWV (SEQ ID NO: 24)
(5) A monoclonal antibody or an antigen-binding fragment thereof, comprising a heavy chain CDR1, a heavy chain CDR2, a heavy chain CDR3, a light chain CDR1, a light chain CDR2, and a light chain CDR3 having 60% or more sequence identity with the respective heavy chain CDR1, heavy chain CDR2, heavy chain CDR3, light chain CDR1, light chain CDR2, and light chain CDR3 recited in any one of (1) to (4), which monoclonal antibody or an antigen-binding fragment thereof undergoes an antigen-antibody reaction with SARS-CoV-2.
(6) An immunoassay method for SARS-CoV-2, comprising performing an immunoassay of SARS-CoV-2 utilizing an antigen-antibody reaction between the monoclonal antibody or the antigen-binding fragment thereof according to any one of (1) to (5) and SARS-CoV-2 in a sample.
(7) The method according to (6), wherein the immunoassay method is a sandwich method, and the monoclonal antibody or the antigen-binding fragment thereof is used for at least one of labeling or solid phase.
(8) An immunoassay device for SARS-CoV-2, comprising the monoclonal antibody or the antigen-binding fragment thereof according to any one of (1) to (5).

### Effects of the Invention

The present invention provides a monoclonal antibody capable of rapidly, simply, and highly sensitively detecting and measuring SARS-CoV-2 contained in a test sample, and an immunoassay method and immunoassay device for SARS-CoV-2 using the same.

### MODES FOR CARRYING OUT THE INVENTION

Hereinafter, embodiments of the present invention will be described in detail.

### 1. Monoclonal Antibody or Antigen-Binding Fragment Thereof

The monoclonal antibody of the present invention is a monoclonal antibody having each heavy chain CDR1, heavy chain CDR2, heavy chain CDR3, light chain CDR1, light chain CDR2, and light chain CDR3 defined in the following (1) to (4).
(1)
(a-1) heavy chain CDR1 consisting of the amino acid sequence of GYTFTSYWMH (SEQ ID NO: 1),
(b-1) heavy chain CDR2 consisting of the amino acid sequence of YINPTTGYTDYNQKFKD (SEQ ID NO: 2),
(c-1) heavy chain CDR3 consisting of the amino acid sequence of EIYFGDVKTWFAY (SEQ ID NO: 3),
(d-1) light chain CDR1 consisting of the amino acid sequence of STSQDISNYLN (SEQ ID NO: 4),
(e-1) light chain CDR2 consisting of the amino acid sequence of TSNLQSG (SEQ ID NO: 5),
(f-1) light chain CDR3 consisting of the amino acid sequence of QQYSKFPYT (SEQ ID NO: 6)
(2)
(a-2) heavy chain CDR1 consisting of the amino acid sequence of GYTFSNYWIE (SEQ ID NO: 7),
(b-2) heavy chain CDR2 consisting of the amino acid sequence of EILPGSDITNYNEKFKD (SEQ ID NO: 8),
(c-2) heavy chain CDR3 consisting of the amino acid sequence of VYYYPGSLAWFAY (SEQ ID NO: 9),
(d-2) light chain CDR1 consisting of the amino acid sequence of SASQDISNYLN (SEQ ID NO: 10),
(e-2) light chain CDR2 consisting of the amino acid sequence of YTSSLHS (SEQ ID NO: 11),
(f-2) light chain CDR3 consisting of the amino acid sequence of QQYSKLPYT (SEQ ID NO: 12)
(3)
(a-3) heavy chain CDR1 consisting of the amino acid sequence of GYTFSSYWIE (SEQ ID NO: 13),
(b-3) heavy chain CDR2 consisting of the amino acid sequence of EILPGSGNTYYNEKFKG (SEQ ID NO: 14),
(c-3) heavy chain CDR3 consisting of the amino acid sequence of WEWLLRLYGLDY (SEQ ID NO: 15),
(d-3) light chain CDR1 consisting of the amino acid sequence of KASQSVDYDGESYMN (SEQ ID NO: 16),
(e-3) light chain CDR2 consisting of the amino acid sequence of AASNLES (SEQ ID NO: 17),
(f-3) light chain CDR3 consisting of the amino acid sequence of QQSNEDPYT (SEQ ID NO: 18)
(4)
(a-4) heavy chain CDR1 consisting of the amino acid sequence of GFSFSDYFMY (SEQ ID NO: 19),
(b-4) heavy chain CDR2 consisting of the amino acid sequence of TISDGGIYTYYPDSVKG (SEQ ID NO: 20),
(c-4) heavy chain CDR3 consisting of the amino acid sequence of DLDYFGSTIAY (SEQ ID NO: 21),
(d-4) light chain CDR1 consisting of the amino acid sequence of RSSAGALTTRNYAN (SEQ ID NO: 22),
(e-4) light chain CDR2 consisting of the amino acid sequence of GTSNRAP (SEQ ID NO: 23),
(f-4) light chain CDR3 consisting of the amino acid sequence of ALWYSNHWV (SEQ ID NO: 24)

The monoclonal antibody or the antigen-binding fragment thereof of the present invention has a basic structure consisting of a heavy chain and a light chain, each of which has a variable region capable of specifically binding to an antigen. VH refers to the variable region of the heavy chain, and VL refers to the variable region of the light chain. The variable regions of the heavy and light chains each include amino acid sequences of complementarity determining regions (CDRs), namely, CDR1, CDR2, and CDR3, and framework regions (FRs). For example, a variable region includes three CDRs together with three or four FRs (e.g., FR1, FR2, FR3, and optionally FR4).

The monoclonal antibody of the present invention also encompasses four-chain antibodies (e.g., two light chains and two heavy chains), recombinant antibodies, or modified antibodies (e.g., chimeric antibodies, humanized antibodies, human antibodies, CDR-grafted antibodies, primatized antibodies, deimmunized antibodies, synhumanized antibodies, half-antibodies, bispecific antibodies). Further, the class of the monoclonal antibody is not limited to IgG, and may be IgM or IgY.

In the present invention, the antigen-binding fragment of a monoclonal antibody is a fragment in which only the antigen-binding site of the monoclonal antibody is isolated, and examples include fragments having specific antigen-binding properties produced by known methods, such as Fab, Fab′, F(ab′)₂, and single-chain antibodies (scFv).

When producing such antigen-binding fragments, preparation methods known to those skilled in the art can be used, for example, a method involving digesting the antibody with a proteolytic enzyme (e.g., pepsin or papain) by a conventional method, followed by purification using a known protein separation and purification method, or a preparation method by genetic recombination can be exemplified.

As the monoclonal antibody of the present invention described above, a monoclonal antibody having each heavy chain CDR1, heavy chain CDR2, heavy chain CDR3, light chain CDR1, light chain CDR2, and light chain CDR3 in Antibodies 1 to 4 obtained in the Examples below is preferred, from the viewpoint of being able to detect SARS-CoV-2 with even higher sensitivity. Each heavy chain CDR1, heavy chain CDR2, heavy chain CDR3, light chain CDR1, light chain CDR2, and light chain CDR3 of Antibodies 1 to 4 is as shown in Table 2 described later.

Note that a monoclonal antibody comprising a heavy chain CDR1, a heavy chain CDR2, a heavy chain CDR3, a light chain CDR1, a light chain CDR2, and a light chain CDR3 having 60% or more, preferably 65% or more, more preferably 70% or more, still more preferably 75% or more, still more preferably 80% or more, still more preferably 85% or more, still more preferably 90% or more, still more preferably 91% or more, still more preferably 92% or more, still more preferably 93% or more, still more preferably 94% or more, still more preferably 95% or more, still more preferably 96% or more, still more preferably 97% or more, still more preferably 98% or more, still more preferably 99% or more sequence identity with each of the heavy chain CDR1, heavy chain CDR2, heavy chain CDR3, light chain CDR1, light chain CDR2, and light chain CDR3 possessed by each of the above-mentioned Antibodies 1 to 4, and which undergoes antigen-antibody reaction with SARS-CoV-2, is also a monoclonal antibody of the present invention. Here, “sequence identity” refers to the value, expressed as a percentage, obtained by aligning two amino acid sequences such that the number of matching amino acids is maximized (inserting gaps if necessary), and dividing the number of matching amino acids by the total number of amino acids (or the number of amino acids in the longer sequence if the total numbers differ), and can be easily calculated by a well-known software such as BLAST.

### 2 Method for Producing Monoclonal Antibody

The monoclonal antibody of the present invention can be obtained by using a known immunological technique, immunizing an animal to be immunized with a complex or extract containing the target antigen SARS-CoV-2, or with SARS-CoV-2 or partial peptides thereof, and producing hybridomas using cells from the immunized animal. The length of the peptide used for the immunization is not particularly limited, but preferably a peptide of 5 amino acids or more, more preferably 10 amino acids or more, can be used as the immunogen.

The immunogen can be obtained from a culture medium, but it can also be obtained by incorporating DNA encoding an arbitrary SARS-CoV-2 antigen into a plasmid vector and introducing this into a host cell for expression. An arbitrary SARS-CoV-2 antigen or its partial peptide to be used as an immunogen can also be expressed as a fusion protein with proteins exemplified below, and used as an immunogen after purification or without purification. For the production of fusion proteins, glutathione S-transferase (GST), maltose-binding protein (MBP), thioredoxin (TRX), Nus tag, S tag, HSV tag, FRAG tag, polyhistidine tag, etc., which are generally used by those skilled in the art as “protein expression/purification tags”, can be utilized. It is preferable to use such a fusion protein as an immunogen after cleaving the fusion protein into an arbitrary SARS-CoV-2 antigen or its partial peptide portion and the other tag portion using a digestive enzyme, followed by separation and purification.

Preparation of monoclonal antibodies from immunized animals can be easily performed by the well-known method of Köhler et al. (Kohler et al., Nature, vol. 256, p 495-497 (1975)). That is, antibody-producing cells such as spleen cells or lymphocytes are collected from the immunized animal, fused with mouse myeloma cells by a conventional method to produce hybridomas, the obtained hybridomas are cloned by a method such as limiting dilution, and among the monoclonal antibodies produced by each cloned hybridoma, monoclonal antibodies that undergo an antigen-antibody reaction with the antigen used for immunizing the animal are selected.

Purification of monoclonal antibodies from ascites fluid or culture supernatant can be performed using known immunoglobulin purification methods. Examples include fractionation methods by salting out using ammonium sulfate or sodium sulfate, PEG fractionation methods, ethanol fractionation methods, DEAE ion-exchange chromatography methods, and gel filtration methods. Furthermore, purification is also possible by affinity chromatography using a carrier to which Protein A, Protein G, or Protein L is bound, depending on the animal species immunized and the class of the monoclonal antibody.

Moreover, the monoclonal antibody used in the present invention can be produced by genetically modified plants. Such antibodies can be produced using a plant transient expression system.

The monoclonal antibody used in the present invention can also be obtained as a recombinant product using mammalian cells as the expression host. Examples of mammalian cells in this case include CHO (Chinese Hamster Ovary) cells and HEK293 (Human Embryonic Kidney cells 293) cells, but are not limited thereto. Furthermore, regarding the method for obtaining it as a recombinant product, examples thereof include transient expression systems using plasmid vectors or viral vectors lacking autonomous replication ability, semi-stable expression systems using episomal vectors provided with a nuclear localization signal and having autonomous replication ability, and stable expression systems in which the target gene is inserted into the genome of the expression host, and are not particularly limited.

For example, in the above-mentioned Antibodies 1 to 4, since the amino acid sequences of the heavy chain CDR1, heavy chain CDR2, heavy chain CDR3, light chain CDR1, light chain CDR2, and light chain CDR3 are specified, it is also possible to produce monoclonal antibodies having the same six types of CDR regions, i.e., having the same binding affinity, by genetic engineering techniques by expressing a gene incorporating polynucleotides encoding these six types of CDRs. Such techniques are well-known and described, for example, in JP 6614523 B.

### 3. Immunoassay Method

Except for using the above-described monoclonal antibody of the present invention or its antigen-binding fragment, any well-known immunoassay method can be adopted, and each immunoassay method itself can be performed in the same manner as conventionally.

In the present invention, by using the above-mentioned monoclonal antibody or its antigen-binding fragment, SARS-CoV-2 can be detected with extremely high sensitivity. Hereinafter, in the description up to the Examples, unless otherwise clear from the context, “monoclonal antibody” means “monoclonal antibody or its antigen-binding fragment”.

In the present invention, the detection of SARS-CoV-2 is performed by immunoassaying SARS-CoV-2 utilizing the antigen-antibody reaction between the above-mentioned monoclonal antibody and SARS-CoV-2 in the sample. The phrase “monoclonal antibody undergoes antigen-antibody reaction with SARS-CoV-2” means that the monoclonal antibody specifically reacts with SARS-CoV-2. “Specifically” means that in a system where the antigen protein and the monoclonal antibody are mixed, the antibody does not cause an antigen-antibody reaction at a detectable level with proteins other than the target protein component in the antigen, or even if the antibody and coexisting substances other than the antigen cause some kind of binding reaction or association reaction with the antibody or antigen, it only causes a reaction clearly weaker than the antigen-antibody reaction of the antibody with the antigen.

In the present invention, as the immunoassay method, any method well-known to those skilled in the art, such as competitive methods, agglutination methods, Western blotting methods, immunostaining methods, and sandwich methods, can be used.

As the immunoassay method of the present invention, the sandwich method is preferred. In the sandwich method, a complex is formed by sandwiching the antigen with two antibodies, and the complex is detected. The sandwich method itself is well-known in the field of immunoassay, and can be performed, for example, by immunochromatography or ELISA methods. These sandwich methods themselves are all well-known, and the method of the present invention can be performed by a well-known sandwich method, except for using the above-mentioned monoclonal antibody or its antigen-binding fragment.

In the sandwich method, one or two or more types of antibodies (an antibody immobilized on a solid phase and a labeled antibody) that recognize the antigen are used. When two or more types of antibodies are used, at least one of these two types of antibodies is the above-mentioned monoclonal antibody. Also, a complex may be formed by sandwiching the antigen with the same two antibodies. Preferably, one or two types of antibodies that recognize the above peptide are used.

In immunoassays based on the sandwich method detection principle, as the solid phase on which the antibody is immobilized, anything that can immobilize the antibody by known techniques can be used, and for example, known materials such as porous thin films (membranes) having capillary action, particulate matter, test tubes, and resin plates can be arbitrarily selected. As the substance for labeling the antibody, enzymes, radioisotopes, fluorescent substances, luminescent substances, colored particles, colloidal particles, etc., can be used. Among the immunoassay methods using the various materials mentioned above, the immunochromatography method, which is a lateral flow type immunoassay method using a membrane, is particularly preferred from the viewpoint of simplicity and rapidity of clinical testing.

In the present invention, even when quantifying or semi-quantifying SARS-CoV-2 using the monoclonal antibody, since quantification and semi-quantification inevitably involve “measurement,” they are included in the “measurement” in the present invention. That is, in the present invention, the “measurement” in immunoassay includes quantification, semi-quantification, and detection.

The present invention also provides an immunoassay device capable of performing lateral flow immunoassay using the above monoclonal antibody. The immunoassay device provided by the present invention comprises a support having a detection region where an antibody (antibody 1) for capturing the analyte (antigen) is immobilized, a label region having a movable labeled antibody (antibody 2), a sample pad for dropping the sample, an absorption zone for absorbing the developed sample solution, and a backing sheet for laminating these members together, wherein at least one of antibody 1 and antibody 2 is the above monoclonal antibody of the present invention. This immunoassay device is also called an immunochromatographic test strip. Note that multiple monoclonal antibodies can be used for the support having a detection region on which an antibody (antibody 1) capturing the analyte (antigen) is immobilized, and as the movable labeled antibody (antibody 2). Also, a polyclonal antibody can be combined.

The support is a material having the ability to immobilize the antibody for capturing the substance to be detected (antigen), and also having the ability not to hinder the horizontal passage of liquid. Preferably, it is a porous thin film having capillary action, and is a material capable of transporting liquid and components dispersed therein by absorption. The material constituting the support is not particularly limited, and examples include cellulose, nitrocellulose, cellulose acetate, polyvinylidene difluoride (PVDF), glass fiber, nylon, polyketone, etc. Among these, those made into a thin film using nitrocellulose are more preferable. A membrane on which an antibody is immobilized is called an antibody-immobilized membrane.

The label region is composed of a porous substrate containing a labeled antibody, and commonly used materials such as glass fiber or non-woven fabric can be used as the substrate material. The substrate is preferably in the form of a pad with a thickness of about 0.3 mm to 0.6 mm in order to impregnate a large amount of labeled antibody. The porous substrate impregnated with the labeled antibody and dried is also called a dry pad.

For labeling the labeled antibody, enzymes such as alkaline phosphatase and horseradish peroxidase, metal colloids such as gold colloid, silica particles, cellulose particles, colored polystyrene particles, and colored latex particles are often used. When using colored particles such as metal colloid particles, colored polystyrene particles, or colored latex particles, coloring occurs due to the aggregation of these labeling reagents, so this coloring is measured. Particles on which antibodies are immobilized are called antibody-immobilized particles. The amount of antibody immobilized is not particularly limited, but it is sufficient if several ng to several tens of μg are present in the labeling region.

The detection region refers to a part of the support where the antibody that captures the substance to be detected (antigen) is immobilized. The detection region is provided with at least one region where the antibody for capturing the antigen is immobilized. The detection region only needs to be included in the support, and the antibody should be immobilized on the support. The amount of the antibody immobilized is not particularly limited, but it is sufficient if several ng to several tens of μg are immobilized in the detection region.

The sample pad is a site for applying the sample and is a porous material. The sample pad is the most upstream site of the immunoassay device. Common filter paper, glass fiber, non-woven fabric, etc., can be used for this material. To use a large amount of sample for immunoassay, it is preferably in the form of a pad with a thickness of about 0.3 mm to 1 mm. The sample also includes samples prepared using the specimen, such as samples obtained by suspending the specimen in another solution.

The absorption zone is a member for absorbing components supplied to the support that were not involved in the reaction in the detection region. For this material, highly water-retentive filter paper, sponge, etc., made of a common natural polymer compound, synthetic polymer compound, etc., can be used, but those with high water absorbency are preferable for promoting the development of the sample.

The backing sheet is a member to which all the aforementioned materials, namely the support, sample pad, label region, absorption zone, etc., are attached and fixed with partial overlap. The backing sheet is not necessarily required if these materials can be arranged and fixed at optimal intervals, but it is generally preferable to use it for convenience in manufacturing or use.

The immunoassay device of the present invention may further include a control display region (member). The control display region is a site indicating that the test was performed correctly. For example, the control display region exists downstream of the detection region, and emits a signal by coloring etc. when the sample reaches the control display region after passing through the detection region. In the control display region, a substance that binds to the antibody bound to the labeled carrier may be immobilized, or a reagent such as a pH indicator that changes color when the sample arrives may be immobilized. When the antibody bound to the labeled carrier is a mouse monoclonal antibody, an anti-mouse IgG antibody may be used.

The size of the immunoassay device is not limited, but for example, it is about several cm to ten-odd cm in length and several mm to several cm in width.

The immunoassay device of the present invention may be housed in a storage container, which can prevent deterioration due to, for example, ultraviolet rays or moisture in the air. Also, when using contaminated or infectious samples, the storage container can prevent the testing personnel performing the assay from being contaminated or infected. For example, a resin case of an appropriate size can be used as the storage container, and the device of the present invention can be stored in the case. The storage container and the immunoassay device housed therein may be collectively referred to as an immunoassay device.

The present invention can provide a kit including the SARS-CoV-2 immunoassay device containing the above monoclonal antibody. The kit may further include a brochure, a sample collection tool, etc.

In the method of the present invention, a complex of antibody 2, which can bind to the substance to be detected (labeled reagent) labeled with a suitable labeling substance such as colored polystyrene particles or gold colloid, and the substance to be detected is developed and moved on a solid phase support on which antibody 1 is immobilized, utilizing capillary action. As a result, a complex of immobilized substance-substance to be detected-labeled reagent is formed on the solid phase support, and by detecting the signal of the labeled reagent emitted from the complex (in the case of gold colloid, the solid phase support portion where the substance capable of binding to the substance to be detected is immobilized turns red), the substance to be detected can be detected. The immunoassay method can be performed at 5-30° C., preferably at room temperature.

Note that the number of detection regions and the types of labeled antibodies included in the label region are not limited to one, and by using antibodies corresponding to multiple analytes, two or more types of antigens can be detected with the same immunoassay device.

By the method of the present invention, it is possible to detect whether or not one is infected with SARS-CoV-2, and if a SARS-CoV-2 antigen is detected in the test sample, it can be determined that one is infected with SARS-CoV-2.

When the above antibody of the present invention is used, SARS-CoV-2 can be specifically recognized, and the above monoclonal antibody does not recognize other viruses, such as Adenovirus, Coxsackievirus, Echo virus, Herpes simplex virus, Human Metapneumovirus, Influenza virus, Measles virus, Mumps virus, Parainfluenza virus, Respiratory syncytial virus infection (RS virus), etc., and does not erroneously detect these viruses.

Examples of the test sample include biological samples that may contain SARS-CoV-2 proteins, such as body fluids like human or animal blood, serum, plasma, urine, semen, cerebrospinal fluid, saliva, sweat, tears, ascites, or amniotic fluid; mucus; feces; organs such as blood vessels or liver; tissues; cells, or extracts thereof. Preferred are cells and secretions from the oral cavity, tonsils, nasal cavity, pharynx, larynx, trachea, bronchi, or lungs, which are easy to collect, nasal swabs, pharyngeal swabs, gargle solutions, sputum, tracheal aspirates, bronchoalveolar lavage fluid, saliva, etc.

The method for collecting these samples is not particularly limited, and known methods can be adopted. Specifically, methods using cotton swabs can be mentioned.

### EXAMPLES

Hereinafter, the present invention will be described more specifically based on examples. However, the present invention is not limited to the following examples.

### Example 1: Production of Monoclonal Antibody Recognizing N Protein of SARS-CoV-2

### 1. Preparation of SARS-CoV-2 N Protein Antigen

DNA encoding SARS-CoV-2 N protein was expressed in E. coli using an expression vector, cultured for several days, and then the protein was purified and used.

### 2. Production of Anti-SARS-CoV-2 N Protein Monoclonal Antibody

The SARS-CoV-2 N protein antigen from 1 was used to immunize BALB/c mice. After rearing for a certain period, iliac lymph nodes were excised from the mice. Multiple hybridoma cell lines producing anti-SARS-CoV-2 N protein antibodies were obtained by the “mouse iliac lymph node method” (Sado Y et al., Acta Histochem. Cytochem. 39:89-94 (2006)).

The obtained cell lines were administered intraperitoneally to pristane-treated BALB/c mice, and after about 2 weeks, antibody-containing ascites was collected. IgG was purified from the obtained ascites by affinity chromatography using a Protein A column, yielding multiple purified anti-SARS-CoV-2 N protein monoclonal antibodies (hereinafter sometimes referred to as “anti-N protein antibodies”).

In the following examples, among the multiple obtained anti-SARS-CoV-2 N protein monoclonal antibodies, four antibodies, Antibodies 1 to 4, selected considering reactivity and specificity, were used.

### Example 2: Immunoassay Device for Measuring SARS-CoV-2

1. Immobilization of Anti-SARS-CoV-2 N Protein Antibody onto Nitrocellulose Membrane

Solutions prepared by diluting the anti-N protein antibodies separated in Example 1 with a buffer, and anti-mouse IgG antibody were provided. The anti-N protein antibody was applied linearly to the sample pad side of a nitrocellulose membrane backed with a PET film, and the anti-mouse IgG antibody was applied linearly to the absorption zone side. Thereafter, the nitrocellulose membrane was thoroughly dried under warm air to obtain an anti-N protein antibody-immobilized membrane.

2. Immobilization of Anti-SARS-CoV-2 N Protein Antibody onto Colored Polystyrene Particles

The anti-N protein antibody prepared in Example 1 was bound to colored polystyrene particles, suspended in buffer, and sufficiently dispersed by ultrasonic treatment to obtain anti-N protein antibody-bound colored polystyrene particles. Hereinafter referred to as anti-N protein antibody-immobilized particles.

### 3. Application and Drying of Anti-SARS-CoV-2 N Protein Antibody-Bound Colored Polystyrene Particles

A predetermined amount of the anti-N protein antibody-immobilized particles prepared in 2 was applied to a glass fiber non-woven fabric and thoroughly dried under warm air. Hereinafter referred to as the label pad.

### 4. Production of SARS-CoV-2 Test Device

The anti-N protein antibody-immobilized membrane prepared in 1 and the label pad prepared in 2 and 3 were laminated with other members (backing sheet, absorption zone, sample pad) and cut into 5 mm widths to obtain a SARS-CoV-2 test device.

### 5. Confirmation of Specificity and Accuracy of SARS-CoV-2 Test Device

To the SARS-CoV-2 test device prepared in 4, 50 μL of buffer (10 mM Tris (pH7.0), 1% (w/v) polyoxyethylene octylphenyl ether, 3% (w/v) arginine, 3% (w/v) BSA) containing viruses that cause respiratory infections was added dropwise and allowed to stand for 8 minutes.

A positive result was determined when coloration was visually confirmed at both the application positions of the anti-mouse IgG antibody and the anti-N protein antibody. A negative result was determined when coloration was visually confirmed only at the application position of the anti-mouse IgG antibody, and no coloration was visually confirmed at the application position of the anti-N protein antibody. Some of the judgment results for combinations of each monoclonal antibody are shown in Table 1. Positive results were judged as +++, ++, +, ± in descending order of color intensity, and negative results were judged as −.

TABLE 1

| | Kit 1 | Kit 2 |
|---|---|---|
| No antigen | − | − |
| SARS-CoV-2 N protein antigen | +++ | +++ |
| SARS-CoV-2 inactivated antigen | ++ | ++ |
| 229E inactivated antigen | − | ± |
| OC43 inactivated antigen | − | − |
| HKU1 inactivated antigen | − | − |
| NL63 inactivated antigen | − | − |
| MARS inactivated antigen | − | − |
| SARS-CoV-1 inactivated antigen | ± | ± |

Note)
Kit 1 used antibody 3 bound to the membrane and antibody 1 bound to the colored particles. Kit 2 used antibody 2 bound to the membrane and antibody 4 bound to the colored particles.

### Example 3: Variable Region Analysis of Anti-SARS-CoV-2 N Protein Monoclonal Antibody

### 1. Base Sequence Analysis of Variable Region of Anti-SARS-CoV-2 N Protein Monoclonal Antibody

The hybridoma cell lines producing anti-SARS-CoV-2 N protein monoclonal antibodies 1 to 4 prepared in Example 1 were sent to BioPeak Co., Ltd. to request base sequence analysis of the antibody variable regions. At BioPeak Co., Ltd., RNA was extracted from the hybridoma cell lines, and base sequences were obtained from the amplification products of the anti-SARS-CoV-2 N protein monoclonal antibody variable regions obtained by PCR.

### 2. Prediction of Amino Acid Sequence of Variable Region of Anti-SARS-CoV-2 N Protein Monoclonal Antibody

From the base sequence information obtained in 1, the amino acid sequence of the variable region was predicted using the Kabat numbering system. The results are shown in Table 2.

TABLE 2

| | | | |
|---|---|---|---|
| Antibody 1 | heavy | CDR1sequence | GYTFTSYWMH (SEQ ID NO: 1) |
| | chain | CDR2sequence | YINPTTGYTDYNQKFKD (SEQ ID NO: 2) |
| | | CDR3sequence | EIYFGDVKTWFAY (SEQ ID NO: 3) |
| | light | CDR1sequence | STSQDISNYLN (SEQ ID NO: 4) |
| | chain | CDR2sequence | TSNLQSG (SEQ ID NO: 5) |
| | | CDR3sequence | QQYSKFPYT (SEQ ID NO: 6) |
| Antibody2 | heavy | CDR1sequence | GYTFSNYWIE (SEQ ID NO: 7) |
| | chain | CDR2sequence | EILPGSDITNYNEKFKD (SEQ ID NO: 8) |
| | | CDR3sequence | VYYYPGSLAWFAY (SEQ ID NO: 9) |
| | light | CDR1sequence | SASQDISNYLN (SEQ ID NO: 10) |
| | chain | CDR2sequence | YTSSLHS (SEQ ID NO: 11) |
| | | CDR3sequence | QQYSKLPYT (SEQ ID NO: 12) |
| Antibody3 | heavy | CDR1sequence | GYTFSSYWIE (SEQ ID NO: 13) |
| | chain | CDR2sequence | EILPGSGNTYYNEKFKG (SEQ ID NO: 14) |
| | | CDR3sequence | WEWLLRLYGLDY (SEQ ID NO: 15) |
| | light | CDR1sequence | KASQSVDYDGESYMN (SEQ ID NO: 16) |
| | chain | CDR2sequence | AASNLES (SEQ ID NO: 17) |
| | | CDR3sequence | QQSNEDPYT (SEQ ID NO: 18) |
| Antibody4 | heavy | CDR1sequence | GFSFSDYFMY (SEQ ID NO: 19) |
| | chain | CDR2sequence | TISDGGIYTYYPDSVKG (SEQ ID NO: 20) |
| | | CDR3sequence | DLDYFGSTIAY (SEQ ID NO: 21) |
| | light | CDR1sequence | RSSAGALTTRNYAN (SEQ ID NO: 22) |
| | chain | CDR2sequence | GTSNRAP (SEQ ID NO: 23) |
| | | CDR3sequence | ALWYSNHWV (SEQ ID NO: 24) |

1. A monoclonal antibody or an antigen-binding fragment thereof, comprising heavy chain CDR1-CDR3 of the following (a-1) to (c-1) and light chain CDR1-CDR3 of the following (d-1) to (f-1):
(a-1) heavy chain CDR1 consisting of the amino acid sequence of GYTFTSYWMH,
(b-1) heavy chain CDR2 consisting of the amino acid sequence of YINPTTGYTDYNQKFKD,
(c-1) heavy chain CDR3 consisting of the amino acid sequence of EIYFGDVKTWFAY,
(d-1) light chain CDR1 consisting of the amino acid sequence of STSQDISNYLN,
(e-1) light chain CDR2 consisting of the amino acid sequence of TSNLQSG,
(f-1) light chain CDR3 consisting of the amino acid sequence of QQYSKFPYT.

2. A monoclonal antibody or an antigen-binding fragment thereof, comprising heavy chain CDR1-CDR3 of the following (a-2) to (c-2) and light chain CDR1-CDR3 of the following (d-2) to (f-2):
(a-2) heavy chain CDR1 consisting of the amino acid sequence of GYTFSNYWIE,
(b-2) heavy chain CDR2 consisting of the amino acid sequence of EILPGSDITNYNEKFKD,
(c-2) heavy chain CDR3 consisting of the amino acid sequence of VYYYPGSLAWFAY,
(d-2) light chain CDR1 consisting of the amino acid sequence of SASQDISNYLN,
(e-2) light chain CDR2 consisting of the amino acid sequence of YTSSLHS,
(f-2) light chain CDR3 consisting of the amino acid sequence of QQYSKLPYT.

3. A monoclonal antibody or an antigen-binding fragment thereof, comprising heavy chain CDR1-CDR3 of the following (a-3) to (c-3) and light chain CDR1-CDR3 of the following (d-3) to (f-3):
(a-3) heavy chain CDR1 consisting of the amino acid sequence of GYTFSSYWIE,
(b-3) heavy chain CDR2 consisting of the amino acid sequence of EILPGSGNTYYNEKFKG,
(c-3) heavy chain CDR3 consisting of the amino acid sequence of WEWLLRLYGLDY,
(d-3) light chain CDR1 consisting of the amino acid sequence of KASQSVDYDGESYMN,
(e-3) light chain CDR2 consisting of the amino acid sequence of AASNLES,
(f-3) light chain CDR3 consisting of the amino acid sequence of QQSNEDPYT.

4. A monoclonal antibody or an antigen-binding fragment thereof, comprising heavy chain CDR1-CDR3 of the following (a-4) to (c-4) and light chain CDR1-CDR3 of the following (d-4) to (f-4):
(a-4) heavy chain CDR1 consisting of the amino acid sequence of GFSFSDYFMY,
(b-4) heavy chain CDR2 consisting of the amino acid sequence of TISDGGIYTYYPDSVKG,
(c-4) heavy chain CDR3 consisting of the amino acid sequence of DLDYFGSTIAY,
(d-4) light chain CDR1 consisting of the amino acid sequence of RSSAGALTTRNYAN,
(e-4) light chain CDR2 consisting of the amino acid sequence of GTSNRAP,
(f-4) light chain CDR3 consisting of the amino acid sequence of ALWYSNHWV.

5. A monoclonal antibody or an antigen-binding fragment thereof, comprising a heavy chain CDR1, a heavy chain CDR2, a heavy chain CDR3, a light chain CDR1, a light chain CDR2, and a light chain CDR3 having 60% or more sequence identity with the respective heavy chain CDR1, heavy chain CDR2, heavy chain CDR3, light chain CDR1, light chain CDR2, and light chain CDR3 recited in claim 1, which monoclonal antibody or an antigen-binding fragment thereof undergoes an antigen-antibody reaction with SARS-CoV-2.

6. An immunoassay method for SARS-CoV-2, comprising performing an immunoassay of SARS-CoV-2 utilizing an antigen-antibody reaction between the monoclonal antibody or the antigen-binding fragment thereof according to claim 1 and SARS-CoV-2 in a sample.

7. The method according to claim 6, wherein the immunoassay method is a sandwich method, and the monoclonal antibody or the antigen-binding fragment thereof is used for at least one of labeling or solid phase.

8. An immunoassay device for SARS-CoV-2, comprising the monoclonal antibody or the antigen-binding fragment thereof according to claim 1.

9. A monoclonal antibody or an antigen-binding fragment thereof, comprising a heavy chain CDR1, a heavy chain CDR2, a heavy chain CDR3, a light chain CDR1, a light chain CDR2, and a light chain CDR3 having 60% or more sequence identity with the respective heavy chain CDR1, heavy chain CDR2, heavy chain CDR3, light chain CDR1, light chain CDR2, and light chain CDR3 recited in claim 2, which monoclonal antibody or an antigen-binding fragment thereof undergoes an antigen-antibody reaction with SARS-CoV-2.

10. A monoclonal antibody or an antigen-binding fragment thereof, comprising a heavy chain CDR1, a heavy chain CDR2, a heavy chain CDR3, a light chain CDR1, a light chain CDR2, and a light chain CDR3 having 60% or more sequence identity with the respective heavy chain CDR1, heavy chain CDR2, heavy chain CDR3, light chain CDR1, light chain CDR2, and light chain CDR3 recited in claim 3, which monoclonal antibody or an antigen-binding fragment thereof undergoes an antigen-antibody reaction with SARS-CoV-2.

11. A monoclonal antibody or an antigen-binding fragment thereof, comprising a heavy chain CDR1, a heavy chain CDR2, a heavy chain CDR3, a light chain CDR1, a light chain CDR2, and a light chain CDR3 having 60% or more sequence identity with the respective heavy chain CDR1, heavy chain CDR2, heavy chain CDR3, light chain CDR1, light chain CDR2, and light chain CDR3 recited in claim 4, which monoclonal antibody or an antigen-binding fragment thereof undergoes an antigen-antibody reaction with SARS-CoV-2.

12. An immunoassay method for SARS-CoV-2, comprising performing an immunoassay of SARS-CoV-2 utilizing an antigen-antibody reaction between the monoclonal antibody or the antigen-binding fragment thereof according to claim 2 and SARS-CoV-2 in a sample.

13. An immunoassay method for SARS-CoV-2, comprising performing an immunoassay of SARS-CoV-2 utilizing an antigen-antibody reaction between the monoclonal antibody or the antigen-binding fragment thereof according to claim 3 and SARS-CoV-2 in a sample.

14. An immunoassay method for SARS-CoV-2, comprising performing an immunoassay of SARS-CoV-2 utilizing an antigen-antibody reaction between the monoclonal antibody or the antigen-binding fragment thereof according to claim 4 and SARS-CoV-2 in a sample.

15. An immunoassay method for SARS-CoV-2, comprising performing an immunoassay of SARS-CoV-2 utilizing an antigen-antibody reaction between the monoclonal antibody or the antigen-binding fragment thereof according to claim 5 and SARS-CoV-2 in a sample.

16. An immunoassay device for SARS-CoV-2, comprising the monoclonal antibody or the antigen-binding fragment thereof according to claim 2.

17. An immunoassay device for SARS-CoV-2, comprising the monoclonal antibody or the antigen-binding fragment thereof according to claim 3.

18. An immunoassay device for SARS-CoV-2, comprising the monoclonal antibody or the antigen-binding fragment thereof according to claim 4.

19. An immunoassay device for SARS-CoV-2, comprising the monoclonal antibody or the antigen-binding fragment thereof according to claim 5.
